(19) United States
(12) Patent Application Publication
(10) Pub. No.: US 2002/0192295 A1
(43) Pub. Date: Dec. 19, 2002

(54) Novel use of phospholipids of vegetable and animal origin in nutritional therapy

(76) Inventor: Yves Ponroy, Saint-Legier (CH)

Correspondence Address: BIERMAN MUSERLIAN AND LUCAS, 600 THIRD AVENUE, NEW YORK, NY, 10016

(21) Appl. No.: 10/212,623

(22) Filed: Aug. 5, 2002

Related U.S. Application Data
(63) Continuation-in-part of application No. 09/623,619, filed on Aug. 31, 2000.
Application No. 09/623,619 is a 371 of international application No. PCT/FR00/00069, filed on Jan. 14, 2000.

(30) Foreign Application Priority Data
Jan. 14, 1999 (FR) 99/00335

(51) Int. Cl. A61K 35/78; A61K 35/12; A61K 31/685

(52) U.S. Cl. 424/522; 514/78; 424/757; 424/755; 424/771; 424/776; 424/768

(57) ABSTRACT

The use of phospholipids of animal origin, rich in unsaturated fatty acids of the n-3 series to produce a suitable dietetic and/or pharmaceutical preparation for improving or restoring good mood, fighting against depressive states, chronic fatigue syndrome and asthenia, as well as for attenuating hyperactivity syndrome which can be associated with vegetable oils rich in unsaturated fatty acids of the n-3 series and/or fish oil, and/or vitamins.

### PRIOR APPLICATIONS

This application is a continuation-in-part of U.S. patent application Ser. No. 09/623,619 filed Aug. 31, 2000, which is a 371 of PCT/FR00/00069 filed Jan. 14, 2000.

The present invention concerns the field of biology and more particularly therapy and nutrition.

The present invention has more particularly as its object the use of phospholipids of natural origin in the form of dietetic preparations or in the form of pharmaceutical compositions with an effect on mood, nervous exhaustion and aggressiveness.

The invention has specifically as its object the use of novel preparations or compositions with the aim of fighting a deficiency in type n-3 fatty acids which are characterized in that they contain phospholipids of animal origin as the active ingredient, rich in docosahexanoic acid (DHA), on their own or mixed with vegetable or animal oils rich in n-3 series fatty acids, added with or admixed with an excipient or an inert non-toxic carrier.

### STATE OF THE ART

Several epidemiological studies have contributed to prove that nervous exhaustion, aggressiveness and depression are linked to a deficiency in n-3 series fatty acids, that is to say mainly DHA and its precursor, alpha linolenic acid (18:3 n-3) (J. R HIBBELN, Dietary polyunsaturated fatty acids and depression, Am. J. Clin. Nutr. 62, 1-9 [1995]).

On the other hand, an excess of n-6 fatty acids, represented by linolenic acid (18.2 n-6) present in vegetable oils, used abundantly in the West (peanuts, sunflower or corn oil), can be considered as at the root of considerable vulnerability to daily stress and to depression. So, communities which consume large quantities of fish rich in DHA and n-3 fatty acids seem to have a lower level of depression. In Japanese fishers villages, depression is practically non-existent (K. HASEGAWA: The epidemiological study of depression in later life, J. Affect. Disord S1, p. 1-6 [1985]. Another study showed that the prevalence of depression is 10 times higher in the U.S.A. and Europe than in Taiwan. This can be explained by the fact that, on one hand, Western people consume small amounts of fish and a large quantity of vegetable oils rich in n-6 and deficient in n-3 fatty acids (corn, sunflower, and peanut oil) and that on the other hand, the Taiwanese consume fish and Soya oil rich in n-3: The changing rate 3105 (1992).

This explanation is reinforced by the observation that post-partum depression is accompanied by a significant drop in the level of plasma DHA (M. GITLIN: Psychiatric syndrome liked to reproductive function in women, Am. J. Psychiatry 46, 1413-1422 [1989]).

Even so, the authors did reveal the fact that the red blood cells of depressive patients have a low content of fatty acids of series n-3 (M. PEET: Depletion of Omega 3 fatty acid levels in red blood cell membranes of depressive patients, Biol. Psychiatry 43, 315-319 [1998]).

From a metabolic point of view, it has been shown in animals that a deficiency in n-3 fatty acids leads to various disturbances in the dopaminergic and serotoninergic neuro-transmitters, which makes it possible to explain the resulting behavioral difficulties: S. DELION: Chronic dietary alpha linolenic acid deficiency alters dopaminergic and serotoninergic neurotransmission in rats, J. Nutr. 124, 2466-2476 (1994).

A nutritional supply of n-3 fatty acids in the form of animal phospholipids rich in DHA, can therefore constitute one of the best approaches for the physiological treatment of depression (M.MAES: Fatty acids, Cytokine and Major depression, Biol. Psychiatry 43, 313-314 [1998)).

Novel uses in therapy and in food of a complex with a base of cerebral phospholipids extracted from porcine brain, to compensate for or slow down the signs of ageing, and more specifically mental deterioration, cerebral hypoxia and neurological ageing have already been described in the French patent 2.721.516.

In this patent, the usefulness of phosphoglycerides linked to two chains of fatty acids, either by an ester function (phosphoglycerides) or by an ether function (plasmalogenes) has particularly been disclosed.

It has been shown moreover that cerebral phospholipids have the peculiarity of being linked to fatty acids whose distribution is very specific and very distinct from that of other phospholipids, especially to those of soya.

It has now been shown that cerebral phospholipids, in much lower doses than those previously described, have found new uses, notably in the field of behavioral problems, in the field of mood related problems and in the field of affectivity problems.

These uses no longer concern elderly people, but rather middle aged people (from 30 to 60 years old and, in particular from 40 to 50 years old) with mood problems and young patients with hyperactivity syndrome. In these conditions, the new compositions with a cerebral phospholipid base do not act on syndromes of mental or nervous deterioration, but rather more on the psychological equilibrium. Other sources of series n-3 fatty acids, like vegetable or animal oils rich in n-3 series fatty acids, like linseed oil, nut oil, rapeseed oil, borage oil, oil of evening primrose or fish oil like tuna oil, sardine oil, halibut oil, or cod oil can be added to the compositions according to the invention.

Clinically the trials conducted on children the age of which varies from 9 to 15 years, with a mean age of about 12 years, have demonstrated the efficiency of the treatment, in the children's attention and abnormal agitation which are statistically significatively improved compared to the base values.

More than 50% of the children have their attention increased with the use of the phospholipids of the invention for about two months.

This method of treating psychological disturbances using product of natural origin, is able to insure a better psycho-physiological calendar and more particularly during the difficult periods of the school spear, namely during the re-opening of the school at the very beginning and examination periods at the end of the school year.

Vitamins and in particular the B vitamins, namely vitamin B1, vitamin B2, vitamin B6, vitamin F, folic acid, nicotinamide, panthotenic acid, their derivatives or salts can be added to the compositions according to the invention.

The compositions according to the invention contain from 10 to 300 mg of specific phospholipids from the brain. They are administered via the digestive route 1 to 4 times per day. They do not manifest any toxicity or phenomena of intolerance.

The compositions moreover contain excipients or diluting agents adapted to administration via the digestive way in the form of plain or coated tablets, chewable tablets, lozenges, pills, powders, gelatinized capsules, capsules or similar products, granules, or flavored or unflavored powders.

In the soft capsules, the brain phospholipids can be dispersed or suspended in fish oil.

The appropriate excipients or diluting agents for these routes can be inert mineral compounds, like calcium carbonate, tricalcium phosphate, magnesium phosphate, alumina, colloidal silica, kaolin, clay, aluminum silicate, calcium silicate or ferric oxide for oral use, or water or aqueous liquids or oils for the liquid forms for example.

The inert carriers can be of an organic nature like starch, dextrine, lactose, cellulose, synthetic cellulose derivatives, alginates, carrageenans, caseinates, fatty acids, waxes or resins.

The carriers for dietary preparation can have a nutritional value or make up a caloric source like casein, milk powder, dextrose, maltodextrines. The compositions can for example be flavored by vanilla or maltol, or sweetened with aspartame or acesulfame.

### EXAMPLE 1

Capsules of porcine brain phospholipids

| | |
|---|---|
| phospholipids of cerebral origin | 300 g |
| lactose | 100 g |
| tricalcium phosphate | 140 g |
| calcium caseinate | 25 g |
| magnesium stearate | 5 g |
| for 1000 capsules | |

### EXAMPLE II

Porcine brain phospholipid sugar coated pills

| | |
|---|---|
| phospholipids of cerebral origin | 10 g |
| vitamin B1 | 0.5 g |
| vitamin B6 | 0.5 g |
| magnesium carbonate | 10 g |
| calcium carbonate | 100 g |
| lactose | 100 g |
| corn starch | 50 g |
| calcium stearate | 20 g |
| for 1000 sugar tablets | |

### EXAMPLE III

Soft capsules of porcine brain phospholipids

| | |
|---|---|
| PLC (porcine brain phospholipids) | 100 g |
| Fish oil | 100 g |
| for 1000 soft capsules | |

### EXAMPLE IV

### Clinical Study of the Compositions According to the Invention

Following preliminary observations, a clinical study was carried out using a double blind control with a placebo in patients complaining of chronic fatigue and asthenia with mood problems. The study was multicentric on two similar groups of adults from 30 to 60 years old. The duration of the experiment was two months with either 4 sugar coated tablets per day of an active ingredient containing only 10 mg of specific brain phospholipids per sugar coated tablet, or 4 sugar coated tablets per day of a placebo.

| Experimental group | Control group |
|---|---|
| 67 patients | 65 patients |
| 47 women, 20 men | 38 women, 27 men |
| average age: 46.7 years | average age: 44.6 average |
| average weight: 65.08 kg | average weight: 64.7 kg |
| cultural level 1.63 | cultural level: 1.85 |
| (1 to 3) | (1 to 3) |

The same distribution within the two groups, of intensity and nature of the behavioral problems spread between: mood, affectivity and mental problems, was noted.

These subjective criteria have given rise to a joint evaluation between the patient and the doctor thanks to a questionnaire. The symptomatic analysis of the obtained results are grouped into 5 items that can be summarized in the table below.

Percentage of improved criteria

| | Experiment group | Control group |
|---|---|---|
| | (phospholipids) | (placebo) |
| Mental function | 77.2% | 32% |
| (intellectual ability) | | |
| Behavior | 80.3% | 43.2% |
| (fatigue, energy) | | |
| Mood (depressive) | 74.2% | 52.6% |
| Affectivity | 75% | 41.9% |
| (withdrawal, isolation) | | |
| Minor neurological problems | 76% | 29.7% |
| (tiredness, vertigo) | | |

All the results are significative. All of these results show a statistically significative improvement in the specific phospholipids of the brain group, rich in DHA in comparison to the placebo group.

The tolerance was very good.

### EXAMPLE V

I have studied the clinical and psychometric efficacy of a natural treatment with the compounds of the Examples in 40 children attending school, presenting minor behavioral disorders: agitation and attention troubles.

The group was composed of 25 girls and 15 boys with an average age of 12.09±1.88 years.

At the beginning of the study, the average clinical marks for attention was 2.77±1.07 (from 0=non-attentive to 5=very attentive) and the average clinical marks for agitation was of 1.92±1.44 (from 0=non-agitated to 5 -very agitated).

The composition was administered for 2 months at 4 capsules per day. The results of the treatment show an improvement of the behavior of the patients from a clinical viewpoint. Improvement of Attention is statistically significant, with an increased attention in 57.5% of the subjects. It is more evident in the boys (initially less attentive) than in the girls, but does not depend on the subject's age. Improvement of Agitation is statistically significant, with a decreased agitation in 53.5% of the subjects. It is more marked in the boys (initially more agitated) than in the girls, and it does not depend on the subject's age.

From the psychometric viewpoint, the treatment has allowed us to obtain:

1) During the second phase of the REY test

a statistically significant increase of the average number of recognized symbols. The gain obtained is influenced by the various parameters studied: it is more important in the boys, in the subjects aged 12 and under and in the children presenting more behavior disturbances: weak initial attention and important initial agitation.

I have noted a clear amelioration of the number of recognized symbols in 60% of the subjects.

a statistically significant decrease of the time needed for answering.

80% of the patients were improved, taking into account the increase of the number of recognized symbols and the decease of the answering time, isolated or combined.

2) During the third phase of the REY test

a statistically significant increase of the average number of memorized symbols.

The gain obtained is little influenced by the sex or the age of the subjects. On the other hand, the most important gain has been obtained in the children presenting the most behavior disorders at the beginning of the study.

I have noted a marked betterment of the number of memorized symbols in 68% of the subjects.

An average answering time comparable to the time needed before treatment.

80% of the patients were improved, taking into account the increase of the number of memorized symbols and the decrease of the answering time, combined or isolated.

As a whole, the administration of the composition in children attending school, allows them to obtain a clear betterment of ATTENTION and MEMORY with more marked results when the behavior troubles are initially more important and significantly improved by the treatment. Thus, this treatment of natural origin is able to insure a better psycho-physiological adaptation of the children to the school calendar and more particularly during the difficult periods of the school year: re-opening of the school, examination periods.

Various modifications of the invention may be made without departing from the spirit or scope thereof. It is to be understood that the invention is intended to be limited only as defined in the appended claims.

What I claim is:

1. A method of reducing hyperactivity in young patients comprising administering to young patients an anti-hyperactivity inducing an amount of phospholipids of animal origin rich in non-saturated fatty acids of series n-3 in association with vegetable oil.

2. The method of claim 1 wherein the phospholipids of animal origin are exclusively porcine brain phospholipids.

3. The method of claim 1 wherein the vegetable oil is at least one vegetable oil rich in non-saturated fatty acids of series n-3 selected from the group consisting of nut oil, soya oil, rapeseed oil, linseed oil, borage oil and evening primrose oil.

4. The method of claim 1 wherein the phospholipids also contain fish oil rich in non-saturated fatty acids of series n-3.

5. The method of claim 1 wherein the phospholipids also contain vitamins.

6. The method of claim 1 wherein the phospholipids also contain a member of the group consisting of diluents, excipients and inert carriers.

7. The method of claim 1 wherein the dose of phospholipids is 10 to 300 mg of brain phospholipids.
